(19) United States
(12) Patent Application Publication
(10) Pub. No.: US 2022/0079422 A1
(43) Pub. Date: Mar. 17, 2022

(54) ENDOSCOPE

(71) Applicant: SCIVITA MEDICAL TECHNOLOGY CO., LTD., Suzhou (CN)

(72) Inventor: Yi ZHANG, Suzhou (CN)

(21) Appl. No.: 17/310,987

(22) PCT Filed: Apr. 2, 2019
(86) PCT No.: PCT/CN2019/080956

§ 371 Date: Sep. 3, 2021

(30) Foreign Application Priority Data
Mar. 15, 2019 (CN) 201910198460.2

(51) Int. Cl. A61B 1/005; A61B 1/00

(52) U.S. Cl. CPC A61B 1/0057; A61B 1/0055; A61B 1/00114

(57) ABSTRACT

An endoscope with more flexibility, higher curvature and longer service life, for increasing the convenience of an endoscopic procedure. The endoscope includes a flexible bending section with slits of various width at different parts, which facilitates high dimensional bending of the bending section with the assistance of guide wires, while the wider slits at proximal and distal tips offer higher dimensional bending. Unique shape and structure of the slits relieve bending and twisting stress normally exerted on a spot to a surface, which avoids easy wreckage and thus guarantees a longer service life. Besides, the guide wires fastened through buckles ensure more stability and save all the inconvenience from dispensing glue.

### FIELD OF THE INVENTION

The present invention relates to the internal tube structure in medical appliances. More particularly, the invention relates to an endoscope.

### BACKGROUND

An endoscope is a commonly used medical device, including a control section and an insertion section. The control section includes a housing and a control system inside. The insertion section enters the human body through natural openings or openings formed by surgery and carries out some operations or surgery, under the control of the control section.

Publication NO. CN106455910B discloses a type of bending section and an endoscope equipped with the type of bending section. The width of the slits on the distal tip of the bending section is smaller than the slits on the proximal tip of the bending section. And the spacing between the adjacent slits on the distal tip of the bending section is smaller the spacing of the adjacent slits on the proximal tip of the bending section.

Such intention has a problem in that the curvature is limited and the stress concentration during the bending process can easily damage the tube body. The problem needs to be solved urgently.

### SUMMARY OF THE INVENTION

The invention includes an endoscope, comprising a control section and an insertion section. The insertion section is sequentially connected by a distal tip, a bending section, and a variable diameter insertion tube. The variable diameter insertion tube is connected to the control section. Multiple slits are formed on the wall of the bending section, the width of which gradually and smoothly becomes smaller from the variable diameter insertion tube side to the distal tip of the insertion section. Whereas, at least one slit near the distal tip of the insertion section is wider than the slit next to it but nearer to the variable diameter insertion tube side. By doing that, the invention sets out a new pattern where certain slits with relatively great width are formed at the distal tip of the bending section instead of all the way narrowing of the slits, which can trigger the bending angle of the distal tip of the insertion section to reach as far as ±290°.

Furthermore, the maximum width of two ends of the slits is greater than the width of the middle parts of the slits, and the width values drop smoothly from the highest value position to the middle parts and both ends respectively. The edge contour of the ends of the slits is an arc tangent to the edge contour of the middle parts of the slits. The unique structure expands the contact area of the slit edges during bending, an effective buffer which avoids split of the slit distal tip, and breakage of the bending section during radial twisting.

Furthermore, multiple buckles and grooves are provided on the bending section wall, which ensures firm embedment in the grooves through elastic deformation and coordination of the buckles and grooves. The orthographic projection of the groove is a rectangle with rounded corner, the shape of which can deconcentrate the stress.

Furthermore, inner holes are formed in middle of the buckles, which can be threaded by guide wires. The guide wires are welded with wire end stoppers at the distal tip of the insertion section, which can be wedged against the buckles under the pulling force of the guide wires. The other side of the guide wire is threaded through the variable diameter insertion tube and fixed to the chain drive system of the control section.

Furthermore, the guide wires are in sufficient amount, and can cause the bending section to bend when the guide wires are pulled.

Furthermore, the bending section is coated with an elastic tube on its exterior surface, which is preferably silicone rubber tube or a nylon elastic tube.

Furthermore, the joint between the distal tip of the insertion section and the bending section, and the joint between the bending section 4 and the variable diameter insertion tube are welded by metal tube, which is coated with protective sleeve on its exterior surface.

Furthermore, the protective sleeve is preferably a heat shrinkable tube, which ensures the smoothness of the joint and better sealing of the entire tube body.

Furthermore, working channels, power supply wires and signal wires are deployed inside the tube body of the insertion section.

In summary, the present invention has the following advantages:
1. The present invention provides multiple slits of relatively great width at the distal tip of the bending section which can trigger the bending angle of the distal tip of the insertion section to reach as far as ±290°, through better control of the bending section.
2. Unique shape and structure of the slits relieve bending and twisting stress normally exerted on a spot to a surface, which avoids easy wreckage and thus guarantees a longer service life.
3. The invention provides guide wires firmly fastened by buckles instead of by dispensing glue, whose convenience not only saves the cost, but also ensures effective control of the bending of the bending section.

### BRIEF DESCRIPTION OF THE DRAWINGS

FIG. 1 shows a complete view of an endoscope;

FIG. 2 shows a view of the insertion section of the endoscope;

FIG. 3 shows a view of the bending section in straightened state and bent state;

FIG. 4 shows a cross section view through the A-A section of FIG. 3;

FIG. 5 shows a cross section view through the B-B section of FIG. 3;

FIG. 6 shows a detailed view of the C section of FIG. 3;

FIG. 7 shows a detailed view of the D section of FIG. 3;

FIG. 8 shows a view of the range of the bending angles of the bending section;

FIG. 9 shows a view of the layout of the slits on the bending section;

FIG. 10 shows a view of the semi-circular-arch slits of the bending section in straightened state;

FIG. 11 shows a view of the semi-circular-arch slits of the bending section as it reaches the bending limit;

FIG. 12 shows a view of certain part of the bending section;

FIG. 13 shows a detailed view of the F section in FIG. 12;

FIG. 14 shows an expanded view of the carved surface of certain part of the bending section;

FIG. 15 shows a detailed view of the G section in FIG. 14;

FIG. 16 shows a detailed view of the H section in FIG. 14;

FIG. 17 shows a view of the position of buckles of the bending section;

FIG. 18 shows a cross section view of the interior structure of the insertion section;

FIG. 19 shows a cross section view of the structure of the buckles;

FIG. 20 shows a cross-section view of the control section;

FIG. 21 shows a complete view of the structure of the control section;

FIG. 22 shows a view of the stopper of the control section;

FIG. 23 shows a view of the distal tip of the insertion section.

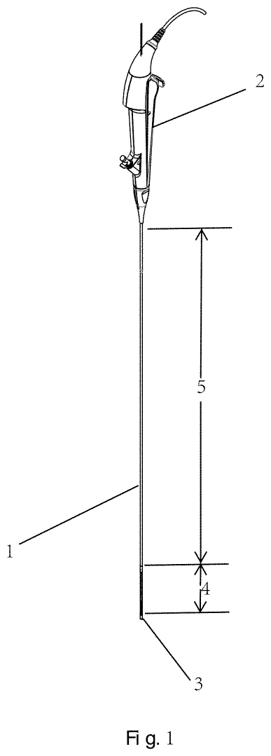

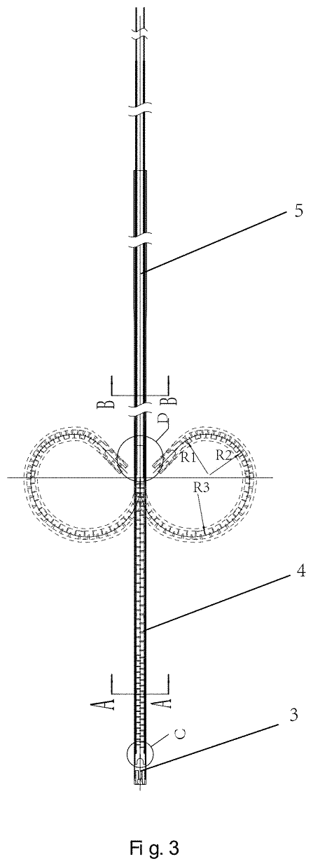

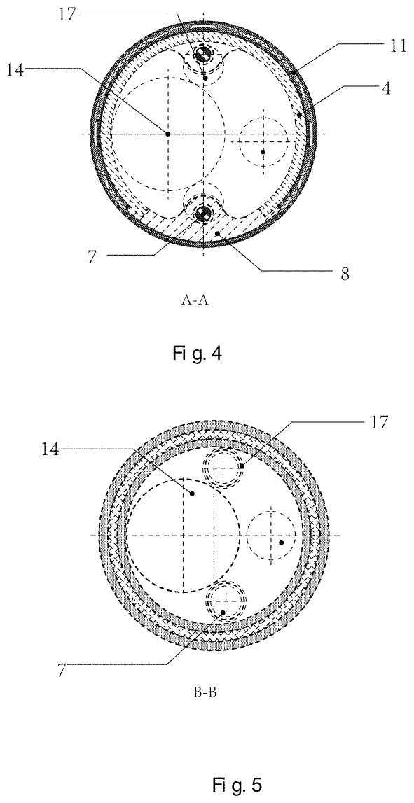

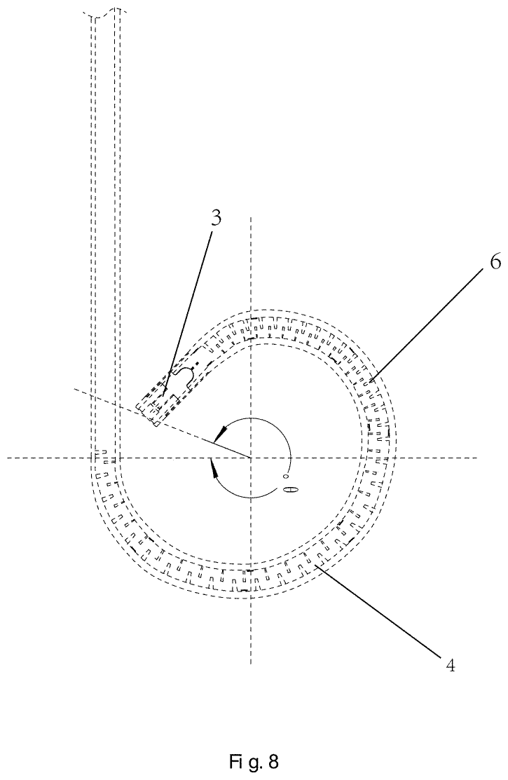

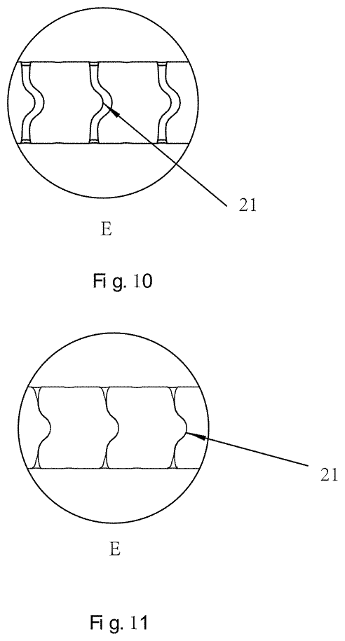

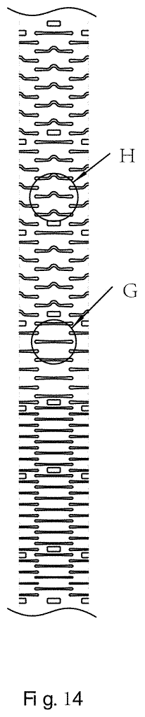

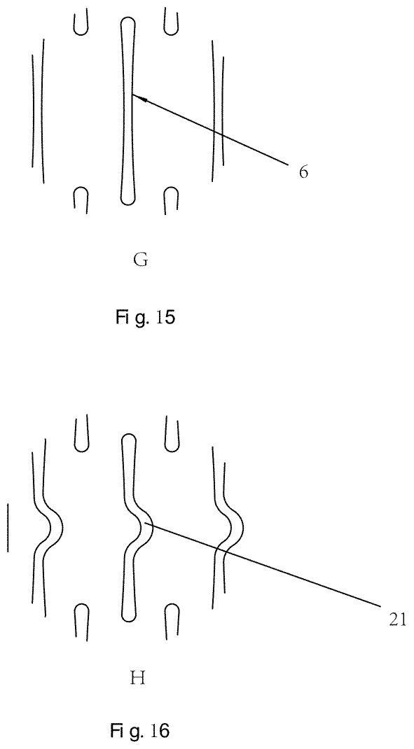

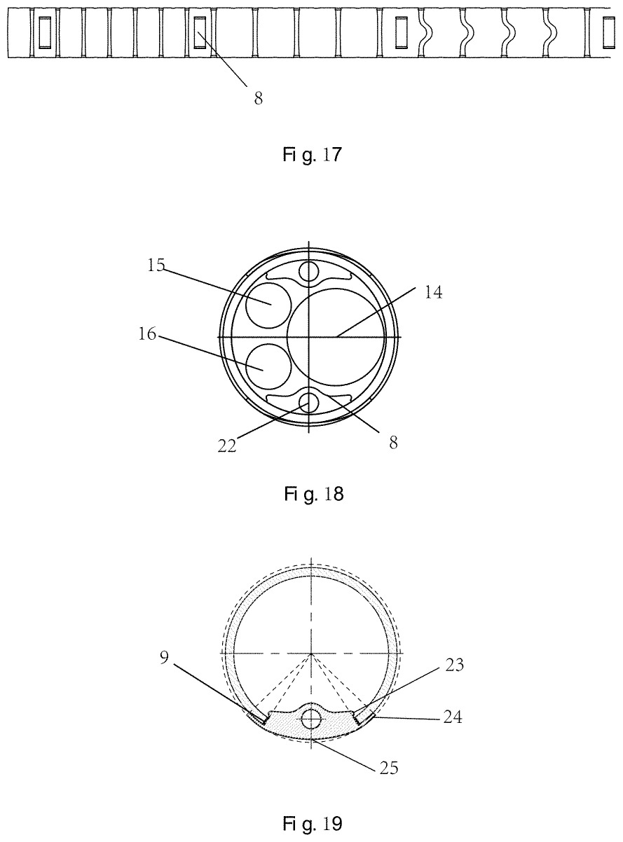

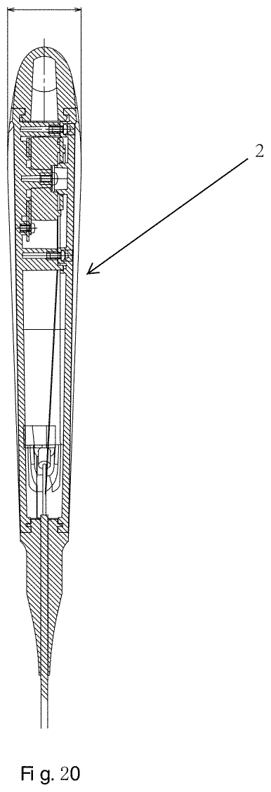

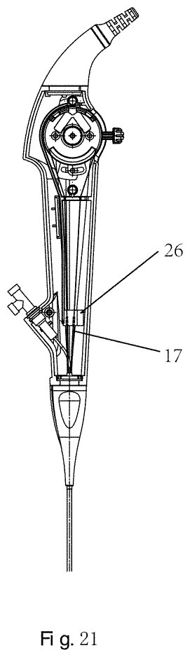

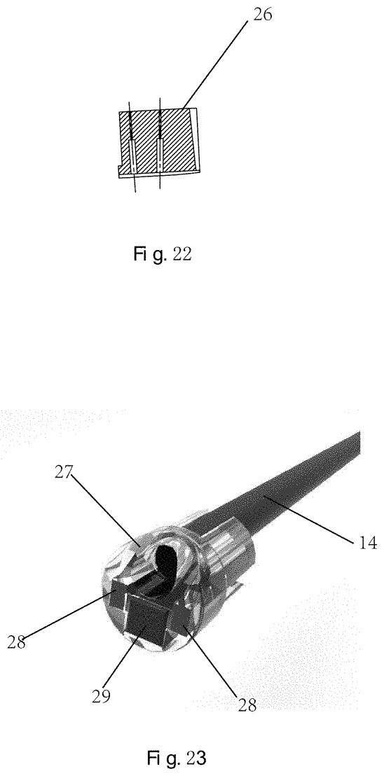

In the FIG. 1 to FIG. 23:

1. Insertion section; 2. Control section; 3. Distal tip of insertion section; 4. Bending section; 5. Variable diameter insertion tube; 6. Slit; 7. Guide wire; 8. Buckle; 9. Groove; 10. End stopper; 11. Elastic tube; 12. Metal tube; 13. Protective sleeve; 14. Working channel; 15. Power supply wire; 16. Signal line; 17. Capillary tube; 18. Start vertebra; 19. Middle vertebra; 20. End vertebra; 21. Semi-circular-arch slit; 22. Inner hole; 23, Buckle protrusion; 24. Buckle tip; 25. Buckle outer surface; 26. Stopper; 27. Transparent tip; 28. LED; 29. COMS.

### DETAILED DESCRIPTION OF THE PREFERRED EMBODIMENTS

Hereinafter, the present invention will be further described in detail with reference to the drawings.

As shown in FIG. 1, an endoscope includes a control section 2 and an insertion section 1, wherein the control section 2 includes a control knob, and the insertion section 1 is sequentially connected by a distal tip 3, a bending section 4, and a variable diameter insertion tube 5, which is connected to the control section 2.

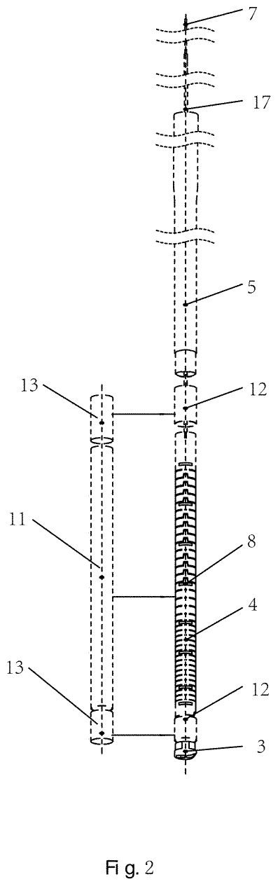

As shown in FIGS. 2, 6 and 7, the bending section 4 is covered with an elastic tube 11, which is preferably a silicone rubber tube or a nylon elastic tube. The joint between the distal tip 3 of the insertion section 1 and the bending section 4, and the joint between the bending section 4 and the variable diameter insertion tube 5 are welded by metal tube 12, which is coated with protective sleeve 13. The protective sleeve 13 is preferably a heat-shrinkable tube, which ensures the smoothness of the joint and better sealing of the entire tube body.

As shown in FIGS. 2, 3, 8 and 9, multiple evenly distributed slits 6 are formed on the wall of the bending section 4, whose width gradually becomes smaller from the variable diameter insertion tube 5 side to the distal tip 3 of the insertion section. At least one slit 6 near the distal tip 3 of the insertion section is wider than the slit 6 next to it but nearer to the variable diameter insertion tube side. The bending section 4 can be subdivided into three parts: the start vertebra 18, the middle vertebra 19, and the end vertebra 20, wherein the start vertebra 18 is connected to the variable diameter insertion tube 5, and the end vertebra 20 is connected to the distal tip 1 of the insertion section. Define the width of the slits 6 on the start vertebra 18 and the middle vertebra 19 as Z, the width of the slits 6 on the end vertebra 20 close to the middle vertebra 19 as Y, and the width of the slits on the end vertebra 20 close to the distal tip 3 of the insertion section as X. Then, Z>Y X>Y. By doing that, the invention sets out a new pattern where certain slits 6 with relatively great width are formed at the distal tip 3 of the bending section 4 instead of all the way narrowing of the slits 6, which can trigger the bending angle θ of the distal tip 3 of the insertion section 1 to reach as far as ±290°. The large bending radius of the start vertebra 18 is R3; the large bending radius of the middle vertebra 19 is R2, which ensures smooth bending; the large bending radius of the end vertebra 20 is R1. The three-section structure ensures a larger bending angle, while the spacing of the slits 6 can be reasonably adjusted according to actual needs for better bending.

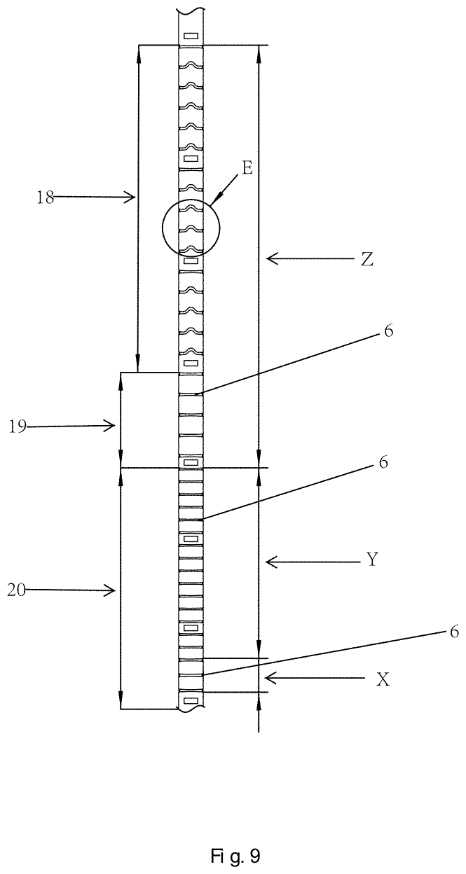

As shown in FIGS. 9, 14 and 15, the slits 6 on the middle vertebra 19 and the end vertebra 20 are elongated along the tube wall, and the maximum width of the two ends of the slits 6 is greater than that of the middle parts of the slits 6. The width value gradually and smoothly decreases from the maximum value position to the middle parts and both ends respectively. The edge contour of the tips of the slits 6 is an arc tangent to the edge contour of the middle parts of the slits 6. The unique structure expands the contact area of the slit 6 edges during bending, an effective buffer which avoids split of the slit 6 distal tip, and breakage of the bending section 4 during radial twisting. As shown in FIGS. 9, 10, 11, 14 and 16, the slits 6 on the start vertebra 18 are specifically formed in the shape of semi-circular arch 21, which structure deconcentrate the pressing stress between the surfaces of contacting parts during limit bending, which effectively avoids distortion. Both the slits 6 and the semi-circular-arch slits 21 are formed through a circular arc streamline carving process.

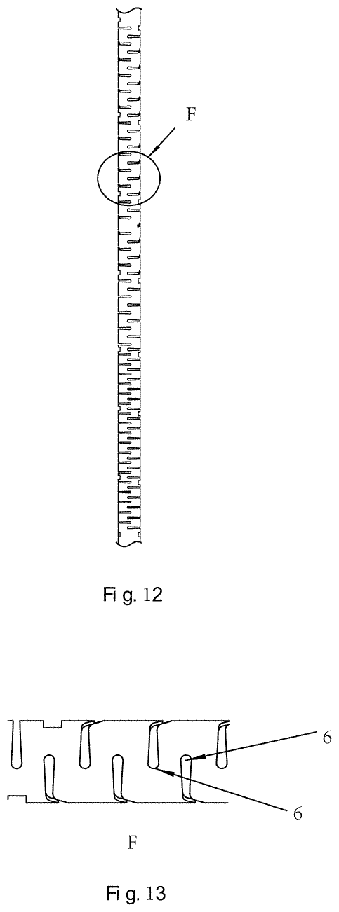

As shown in FIGS. 12 and 13, the slits 6 are distributed in staggered arrangement, and the width of the tips of the slits 6 is about 1.5 times the width of the central parts 6, which avoids breakage of adjacent tips as the bending excesses its limit. The tips of the slits 6 are connected with smoothly rounded corners ensuring that there is no stress concentration during normal bending.

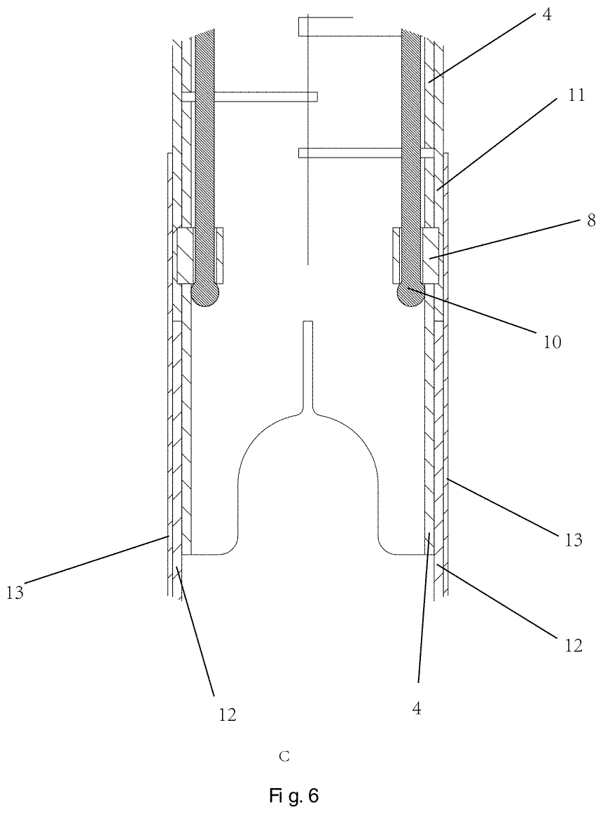

As shown in FIGS. 6, 17, 18 and 19, multiple buckles 8 and grooves 9 are formed on the wall of the bending section 4, which ensures firm embedment in the grooves through elastic deformation and coordination of the buckles 8 and grooves 9. The orthographic projection of the groove 9 is a rectangle with rounded corners, the shape of which can reduce the concentration of stress. Inner holes are formed in middle of the buckles, which can be threaded by guide wires 7. Guide wires 7 are welded with wire end stoppers 10 at the distal tip 3 of the insertion section 1, which can be wedged against the buckles under the pulling force of the guide wires 7, and wherein the other side is threaded through the variable diameter insertion tube 5 and fixed to the chain drive system of the control section 2. In this embodiment, there are two guide wires 7 symmetrically arranged on both sides of the tube body, which can trigger the bending of the bending section 4 when they are pulled. Working channels 14, power supply wires 15 and signal wires 16 are deployed inside the tube body of the insertion section 1.

The buckle 8 is thick in the middle and thin on both sides, comprising a buckle protrusion 23, a buckle tip 24 and an outer surface of the buckle 25. The middle part of the buckle 8 is provided with an inner hole 22 through which the spring tube of the guide wire 7 passes. When assembling, force the buckle 8 into the groove 9 making sure both sides of the buckle 8 are firmly inserted into the groove 9. Both ends of the inner side of the buckle 8 are provided with buckle protrusions 23. Through elastic deformation of the buckle 8 itself, the buckle 8 can be tightly fastened in the groove 9 without falling off. Finally, thread the spring tube of the guide wire 7 through the inner hole 22 of the buckle 8, so that the buckle is firmly fixed in the groove 9. The outer surface 25 of the buckle is connected by multiple arcs, making sure the distance from the center of the arc to the center of the tube is smaller than the distance from the buckle tip 24 to the center of the tube. Such that, the bending section 4 can be more flexible during surgery, because if the side wall of the buckle is too thick, it will protrude on the tube wall, which will affect the operation. In this embodiment, the side wall of the buckle 8 is lighter and thinner and the entire tube body is smoother, which will facilitate the surgery.

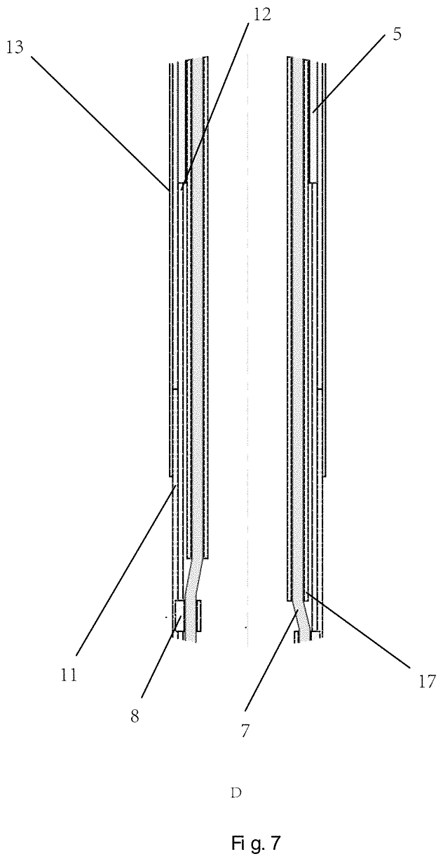

As shown in FIGS. 7, 20, 21 and 22, a layer of capillary tube 17 is provided on the outer layer of the guide wire 7 inside the variable diameter insertion tube 5. The capillary tube 17 is made of stainless steel which protects and limits the guide wire 7. A stopper 26 is fixed inside the control knob, which are provided with two perforations, each divided into two sections. The perforations near the insertion section 1 side are relatively wider, to thread and limit the two capillary tubes 17. The perforations on the other section are relatively narrower for the guide wire 7 to pass through. This structure ensures more accurate, stable, and precise control of the guide wire 7 by the operator, which further contributes to more precise and easy control of the bending section 4.

As shown in FIG. 23, the distal tip 3 of the insertion section 1 in this embodiment is a transparent tip 27, which is provided with LED 28 and COMS 29.

1. An endoscope comprising:
a) a control section 2,
b) an insertion section 1, said insertion section including
a distal tip 3,
a bending section 4, with multiple slits 6 on its wall,
a variable diameter insertion tube 5, connected to the control section 2, and
wherein the width of the slits 6 becoming gradually smaller from the variable diameter insertion tube 5 side to the distal tip 3 of the insertion section 1, wherein, at least one slit 6 near the distal tip 3 of the insertion section 1 is wider than the slit 6 next to it but nearer to the variable diameter insertion tube 5 side.

2. The endoscope according to claim 1, wherein the maximum width of two ends of the slits 6 is greater than the width of the middle parts of the slits 6, and the width values drop smoothly from the highest value position to the middle parts and both ends respectively.

3. The endoscope according to claim 2, wherein the edge contour of the ends of the slits 6 is an arc tangent to the edge contour of the middle parts of the slits 6.

4. The endoscope according to claim 1, wherein multiple buckles 8 and grooves 9 are fixed to the wall of the bending section 4, which ensures firm embedment in the grooves 9 through elastic deformation and coordination of the buckles 8 and grooves 9.

5. The endoscope according to claim 4, wherein inner holes 22 are formed in middle of the buckles 8, which can be threaded by guide wires 7.

6. The endoscope according to claim 5, wherein guide wires 7 are welded with wire end stoppers 10 at the distal tip 3 of the insertion section 1, which can be wedged against the buckles 8 under the pulling force of the guide wires 7, and wherein the other side is threaded through the variable diameter insertion tube 5 and fixed to the chain drive system of the control section 2.

7. The endoscope according to claim 6, wherein the guide wires 7 are in sufficient amount, and can cause the bending section 4 to bend when the guide wires 7 are pulled.

8. The endoscope according to claim 1, wherein the bending section 4 is coated with an elastic tube 11 on its exterior surface.

9. The endoscope according to claim 8, wherein the elastic tube 11 is preferably a silicone rubber tube or a nylon elastic tube.

10. The endoscope according to claim 1, wherein the joint between the distal tip 3 of the insertion section 1 and the bending section 4, and the joint between the bending section 4 and the variable diameter insertion tube 5 are welded by metal tube 12, which is coated with protective sleeve 13 on its exterior surface.

11. The endoscope according to claim 10, wherein the protective sleeve 13 is preferably a heat shrinkable tube, which ensures the smoothness of the joint and better sealing of the entire tube body.

12. The endoscope according to claim 1, wherein working channels 14, power supply wires 15 and signal wires 16 are deployed inside the tube body of the insertion section 1.
